(19) United States
(12) Patent Application Publication
(10) Pub. No.: US 2026/0324572 A1
(43) Pub. Date: Oct. 22, 2026

(54) PLANT TREATMENT FORMULATION

(71) Applicant: Robert Davis Erickson, Wichita, KS (US)

(72) Inventor: Robert Davis Erickson, Wichita, KS (US)

(21) Appl. No.: 19/181,058

(22) Filed: Apr. 16, 2025

(51) Int. Cl. A01H 4/00

(52) U.S. Cl. CPC A01H 4/002; A01H 4/005

(57) ABSTRACT

A micropropagation media for plant tissue cultures has been developed that facilitates superior yields, enhances disease prevention, and provides consistent genetics when used with a variety of plants, particularly Cannibas plants. More particularly, it has been discovered that the micropropagation media described herein can greatly facilitate root, leaf, and shoot growth when used in plant tissue cultures, particularly for Cannibas plants.

### BACKGROUND

### 1. Field of the Invention

The present invention generally relates to a tissue culture media for plants and methods for using such media. More particularly, the present invention generally relates to micropropagation media for cultivating plants and methods of utilizing the micropropagation media.

### 2. Description of the Related Art

Due to more recent legalization efforts, Cannabis plants have become increasingly popular and the market on Cannibas plants grows every year. Cannibas has variety of uses, ranging from medical, industrial, manufacturing, and recreational uses. Cannibas may also be referred to as weed, marijuana, and hemp.

Generally, many plants are traditionally grown using either seeds or stem cuttings. However, these techniques are not applicable to all plants and may not yield the consistent yields and qualities sought by growers or mandated by government regulations.

Thus, tissue culture techniques, such as micropropagation, have been increasingly utilized for commercial plant markets that require a consistent yield of plants with specific genetics, such as Cannibas plants. Tissue culture is a modern plant propagation method that uses small pieces of plant tissue, called explants, which are cultivated in a highly controlled, sterile lab environment. Based on the principle of totipotency, tissue culture allows for the growth of complete plants from small tissue samples, even from a single cell. This method enables the mass production of plants with identical genetic traits. However, these tissue culture techniques generally provide a low yield, may negatively affect cannabinoid content of the cultivated plant, and have little effect on pathogen prevention and control.

Accordingly, there is a need for a superior tissue culture media for plants, particularly Cannibas, which facilitates disease prevention, while also enhancing yield and cannabinoid content of the plant.

### SUMMARY

One or more embodiments of the present disclosure generally concern a method for facilitating plant cultivation. Generally, the method comprises: (a) providing a micropropagation media; (b) providing a sample from a plant; and (c) cultivating the sample in at least a portion of the micropropagation media. In such embodiments, the micropropagation media comprises: (i) water, (ii) at least one tissue culture medium, (iii) at least one gelling agent, (iv) at least one saccharide, (v) a first auxin, and (vi) a second auxin. Furthermore, the first auxin and the second auxin are different auxins are selected from the group consisting of indole-3-acetic acid (“IAA”), indole-3-butyric acid (“IBA”), naphthalene acetic acid (“NAA”), or 2,4-dichlorophenoxyacetic acid (“2,4-D”). In addition, the molar ratio of the first auxin to the second auxin is in the range of about 3:1 to about 1:3.

One or more embodiments of the present disclosure generally concern a micropropagation media for facilitating plant cultivation. Generally, the micropropagation media comprises: (i) water, (ii) at least one tissue culture medium, (iii) a gelling agent, (iv) at least one saccharide, (v) a first auxin, and (vi) a second auxin. Furthermore, the first auxin and the second auxin are different auxins are selected from the group consisting of indole-3-acetic acid (“IAA”), indole-3-butyric acid (“IBA”), naphthalene acetic acid (“NAA”), or 2,4-dichlorophenoxyacetic acid (“2,4-D”). In addition, the molar ratio of the first auxin to the second auxin is in the range of about 3:1 to about 1:3.

One or more embodiments of the present disclosure generally concern a method for facilitating Cannibas cultivation. Generally, the method comprises: (a) providing a micropropagation media; (b) providing a meristem sample from a Cannibas plant; and (c) cultivating the meristem sample in at least a portion of the micropropagation media. In such embodiments, the micropropagation media comprises: (i) water, (ii) a tissue culture medium, (iii) a gelling agent, (iv) 0.5 to 2.5 weight percent of sucrose based on the total weight of the micropropagation media, (v) at least one activated carbon, (vi) indole-3-butyric acid (“IBA”), and (vii) naphthalene acetic acid (“NAA”), wherein the molar ratio of the IBA to the NAA is in the range of about 2:1 to about 1:2.

### DETAILED DESCRIPTION

A micropropagation media for plant tissue cultures has been developed that facilitates superior yields, enhances disease prevention, and provides consistent genetics when used with a variety of plants, particularly Cannibas plants. More particularly, it has been discovered that the micropropagation media described herein can greatly facilitate root, leaf, and shoot growth when used in plant tissue cultures, particularly for Cannibas plants.

It has been discovered that the unique formulation of the micropropagation media described herein facilitates plant growth and prevents the spread of undesirable pathogens and disease amongst the cultivated plants. It is believed that the unique combination and ratio of auxins, along with a specific content of saccharide and other reagents, allows the formation of this unique micropropagation media that exhibits superior properties when utilized for plant tissue cultures. The basic components for forming the micropropagation media are described below, followed by how the media may be produced and utilized.

As previously noted, the combination and ratio of at least two auxins has been observed to provide a synergistic effect in the micropropagation media, particularly in regard to shoot and root formation in Cannibas plants. In one or more embodiments, the micropropagation media may comprise at least two different auxins, e.g., a first auxin and a second auxin. In such embodiments, the auxins may be indole-3-acetic acid (“IAA”), indole-3-butyric acid (“IBA”), naphthalene acetic acid (“NAA”), and/or 2,4-dichlorophenoxyacetic acid (“2,4-D”). In certain embodiments, the first auxin comprises indole-3-butyric acid (“IBA”) and the second auxin comprises naphthalene acetic acid (“NAA”). Furthermore, in certain embodiments, the micropropagation media may comprise a third auxin comprising indole-3-acetic acid (“IAA”) and/or 2,4-dichlorophenoxyacetic acid (“2,4-D”).

It has been discovered that the molar ratio between IBA and NAA can create a synergistic effect that greatly influences plant growth and disease prevention when the micropropagation media is used for tissue cultures. In one or more embodiments, the micropropagation media comprises a molar ratio of the first auxin (e.g., IBA) to the second auxin (e.g., NAA) of less than about 4:1, 3:1, 2.5:1, 2:1, 1.5:1, 1.4:1, 1.3:1, 1.2:1, 1.1:1, or 1:1. Additionally, or alternatively, the micropropagation media comprises a molar ratio of the first auxin (e.g., IBA) to the second auxin (e.g., NAA) of greater than about 1:3, 1:2.5, 1:2, 1:1.5, 1:1.4, 1:1.3, 1:1.2, 1:1.1, or 1:1. In certain embodiments, the micropropagation media comprises a molar ratio of the first auxin (e.g., IBA) to the second auxin (e.g., NAA) in the range of about 3:1 to about 1:3, about 2:1 to about 1:2, about 1.5:1 to about 1:1.5, or about 1:1.

In one or more embodiments, the micropropagation media comprises at least 5, 10, 50, 75, or 100 ppmw (“parts per million by weight”) and/or less than 2,000, 1,000, 500, 400, 300, 200, or 150 ppmw of the first auxin, based on the total weight of the micropropagation media. Additionally, or alternatively, the micropropagation media may comprise at least 5, 10, 50, 75, or 100 ppmw and/or less than 2,000, 1,000, 500, 400, 300, 200, or 150 ppmw of the second auxin, based on the total weight of the micropropagation media. If a third auxin is utilized, the micropropagation media may comprise at least 5, 10, 50, 75, or 100 ppmw and/or less than 2,000, 1,000, 500, 400, 300, 200, or 150 ppmw of the third auxin, based on the total weight of the micropropagation media.

In various embodiments, the micropropagation media may also comprise one or more cytokinins, such as meta-topolin, zeatin, isopenenyladeninie, dihydrozeatin, trans-zeatin, cis-zeatin, kinetin, and/or benzyladenine. In certain embodiments, the cytokinin comprises meta-topolin. If a cytokinin is utilized, the micropropagation media may comprise at least 5, 10, 50, 75, or 100 ppmw and/or less than 2,000, 1,000, 500, 400, 300, 200, or 150 ppmw of one or more cytokinins, based on the total weight of the micropropagation media.

Another important component of the micropropagation media is the saccharide. In one or more embodiments, the micropropagation media comprises a saccharide, such as monosaccharide and/or a disaccharide. In certain embodiments, micropropagation media comprises a disaccharide, such as sucrose. While not wishing to be bound by theory, it has been discovered that certain sucrose contents within the micropropagation media can facilitate rooting during tissue culture; however, the use of too much sucrose has been observed to have negative effects on root formation. Thus, a very specific content of sucrose in the micropropagation media was observed to have a positive influence on root formation during tissue culture. In one or more embodiments, the micropropagation media comprises at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, or 1.9 weight percent of at least one saccharide (e.g., sucrose), based on the total weight of the micropropagation media. Additionally, or alternatively the micropropagation media may comprise less than about 3, 2.9, 2.8, 2.7, 2.6, 2.5, 2.4, 2.3, 2.2, 2.1, 2, 1.9, 1.8, 1.7, 1.6, or 1.5 weight percent of the saccharide (e.g., sucrose), based on the total weight of the micropropagation media.

In addition, the micropropagation media comprises water, such as deionized water. In one or more embodiments, the micropropagation media comprises at least 90, 95, 96, 97, or 98 weight percent of water, based on the total weight of the micropropagation media.

In one or more embodiments, the micropropagation media comprises at least one tissue culture medium. Generally, the tissue culture medium can comprise a Driver and Kuniyuki Walnut (“DKW”) medium and/or a Murashige and Skoog medium. In various embodiments, the culture medium comprises a basal salt mixture containing macronutrients and micronutrients. An exemplary commercial tissue culture medium is C2020 from PhytoTech Labs (Lenexa, KS).

In one or more embodiments, the micropropagation media comprises at least about 0.01, 0.05, 0.1, 0.2, 0.3, 0.4, or 0.5 weight percent and/or less than 2, 1.5, 1, 0.9, 0.8, 0.7, or 0.6 weight percent of the tissue culture medium, based on the total weight of the micropropagation media.

In one or more embodiments, the micropropagation media comprises at least one gelling agent. In such embodiments, the gelling agent comprises agar, agarose, gellan gum, isubgol, or a combination of two or more thereof. In certain embodiments, the gelling agent comprises agar. In various embodiments, the micropropagation media comprises at least about 0.01, 0.05, 0.1, 0.2, 0.3, 0.4, or 0.5 weight percent and/or less than about 2, 1.5, 1, 0.9, 0.8, or 0.7 weight percent of the gelling agent, based on the total weight of the micropropagation media.

In one or more embodiment, the micropropagation media further comprises at least one activated carbon, such as charcoal. The activated carbon may provide coloring to the media, while also acting as an absorbent. In such embodiments, the micropropagation media comprises at least 0.001, 0.005, 0.01, 0.05, 0.1, 0.15, 0.2, 0.25, or 0.3 weight percent and/or less than 1, 0.9, 0.8, 0.7, 0.6, or 0.5 weight percent of the activated carbon (e.g., charcoal), based on the total weight of the micropropagation media.

In one or more embodiments, the micropropagation media further comprises at least one antimicrobial, such as an antibiotic. In such embodiments, the antimicrobial can comprise ampicillin, streptomycin, kanamycin, cefotaxime, or a combination of two or more thereof. In various embodiments, the micropropagation media comprises at least 0.0001, 0.0005, or 0.001 weight percent and/or less than 0.1, 0.05, 0.01, or 0.005 weight percent of the antimicrobial, based on the total weight of the micropropagation media.

Generally, the micropropagation media exhibits a pH that is facilitated for plant cultivation during tissue culture. In one or more embodiments, the micropropagation media exhibits a pH of at least about 3, 3.5, 4, 4.5, 5, 5.5, 5.6, 5.7, or 5.8 and/or less than about 7, 6.9, 6.8, 6.7, 6.6, 6.5, 6.4, 6.3, 6.2, 6.1, 6.0, or 5.9.

Generally, the micropropagation media may be produced by the following methodology: (1) combining at least a portion of the water with the tissue culture medium, saccharide, antimicrobial, and gelling agent under constant agitation; (2) adjusting the pH of the mixture to 5.8 to 6.0; (3) adding the activated carbon to the mixture; (4) adding the remaining water to the mixture; (5) heating the mixture in an autoclave for 15 to 25 minutes, preferably for about 20 minutes, at about 15 psi and a temperature of about 100 to about 135 °C, preferably about 121 °C; (6) cooling the autoclaved mixture; (7) adding the auxins and cytokinins to the cooled autoclave mixture; and (8) pouring the resulting micropropagation media into the individual sterile receptacles of choice (e.g., test tubes, petri dishes, etc.).

The resulting micropropagation media can be used in any tissue culture technique known in the art, such as callus culture, organ culture, meristem culture, embryo culture, or protoplast culture. Such tissue culture techniques can involve: (1) providing the micropropagation media, usually prepared and solidified in the desired sterile receptable (e.g., test tube, plastic boxing, petri dish, etc.); (2) providing a tissue sample from the desired plant; (3) placing the tissue sample within the micropropagation media; and (4) cultivating the tissue sample in at least a portion of the micropropagation media. Generally, the tissue sample from the plants can be any tissue type (e.g., stem, flowers, leaves, roots, etc.) or even a seed from the desired plant. In preferred embodiments, the tissue sample comprises meristematic tissues, such as shoot apical meristem, root apical meristem, intercalary meristem, and/or floral meristem, from the plant. Typically, meristematic tissue samples are preferred due to the high success rate in establishing cultures and their ability to regenerate into whole plants. The cultivation process generally occurs under laboratory conditions where temperature and other environmental factors can be optimized for plant growth.

It has been observed that utilizing meristem tissue samples with the micropropagation media can help mitigate the spread of disease and pathogens amongst the cultivated plants. Generally, meristem culture leverages the rapid cell division at a plant's shoot tip, which often outpaces pathogen spread, to produce disease-free plants. Typically, the tiny meristem tissue is excised and placed onto the sterile, nutrient-rich micropropagation media. The inventive micropropagation media is crucial, as it provides the precise balance of hormones and nutrients necessary for the meristem to develop into a complete plantlet. Without this optimally formulated media, the meristem may fail to thrive, thereby hindering pathogen removal and plant regeneration. Therefore, the success of meristem culture heavily relies on the micropropagation media described herein, which supports healthy plant growth.

Although the micropropagation media has shown to be highly compatible and works great with Cannibas plants, it may also be utilized with other plant varieties with similar success. For instance, the micropropagation media and the process of using it may be utilized with a plant from the Cannibas genus, the Streptocarpus genus, the Chrysanthemum genus, the Musa genus (e.g., bananas), the Fragaria genus, the Solanum genus (e.g., potatoes), the Orchidacae family (e.g., orchids), the Aloe genus, the Rosa genus (e.g., roses), the Petuniagenus, or the Sambucus genus (e.g., elderberry). In certain embodiments, the plant comprises a Cannibas plant, which may be Cannibas sativa, Cannibas indica, or Cannibas ruderalis. Furthermore, the micropropagation media described herein has shown to effectively work with a number of different Cannibas strains, such as Apples and Bananas, Baby Yoda, Butterfingerz, B Scotty, Cherry Pie Kush, Cheetah Piss, Fire Pie, Gaschata, Glue Mintz, Gush Mintz, Jokerz, Permanent Marker, Purple Milk, Purple Urkle, Rainbow Lane, Slurty 3, Wedding Pie #6, and Zechem.

This invention can be further illustrated by the following examples of embodiments thereof, although it will be understood that these examples are included merely for the purposes of illustration and are not intended to limit the scope of the invention unless otherwise specifically indicated.

### EXAMPLES

### Example 1

A micropropagation media was produced in accordance with the following methodology. The base recipe for one liter of the micropropagation media was prepared with 5.32 grams DKW, 6 grams of agar-agar for the gelling agent, 1.5 mL of PPM (Plant Preservative Mixture, a biocide), sucrose (20 grams), 1 gram activated charcoal, and a hormone ratio of 0.9 mM NAA (naphthaleneacetic acid) and 0.8 mM IBA (indole-3-butyric acid). First, 800 mL of distilled water was placed in a beaker on a magnetic stir plate. The culture medium (DKW), sucrose, PPM, and agar were added to the water under constant mixing. The pH of the mixture was adjusted to 5.8-6.0, and then the activated charcoal was added. Afterwards, another 200 mL of distilled water was added and the mixture was autoclaved at 15 psi for 20 minutes. Once cool enough to handle, the hormones (IBA and NAA) were added using filter tips. Subsequently, the formed media was poured into sterile vessels and allowed to solidify.

It was observed that this formulation was able to effectively facilitate root and shoot growth in Cannibas plants during tissue culture.

### Comparative Example 2

A micropropagation media was produced in accordance with formulation in Example 1, instead 30 grams of sucrose was used instead of 20 grams. It was observed that this higher sucrose content in the media failed to induce root formation in the tested Cannibas cultivars. Thus, it was observed that too high of a sucrose content was not desirable or productive in the media.

### DEFINITIONS

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “about” is meant to encompass the stated value±a deviation of not more than 5% of the stated value.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “having,” “has,” and “have” have the same open-ended meaning as “comprising,” “comprises,” and “comprise” provided above.

As used herein, the terms “including,” “include,” and “included” have the same open-ended meaning as “comprising,” “comprises,” and “comprise” provided above.

### NUMERICAL RANGES

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds).

### CLAIMS NOT LIMITED TO DISCLOSED EMBODIMENTS

The preferred forms of the invention described above are to be used as illustration only, and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A method for facilitating plant cultivation, the method comprising:
(a) providing a micropropagation media comprising—
(i) water,
(ii) a tissue culture medium,
(iii) at least one gelling agent,
(iv) at least one saccharide,
(v) a first auxin, and
(vi) a second auxin, wherein the first auxin and the second auxin are different auxins, wherein the first auxin and the second auxin comprise indole-3-acetic acid (“IAA”), indole-3-butyric acid (“IBA”), naphthalene acetic acid (“NAA”), or 2,4-dichlorophenoxyacetic acid (“2,4-D”), wherein a molar ratio of the first auxin to the second auxin is in the range of about 3:1 to about 1:3;
(b) providing a sample from a plant; and
(c) cultivating the sample in at least a portion of the micropropagation media.

2. The method according to claim 1, wherein the first auxin comprises indole-3-butyric acid (“IBA”).

3. The method according to claim 2, wherein the second auxin comprises naphthalene acetic acid (“NAA”).

4. The method according to claim 3, wherein the molar ratio is in the range of about 1.5:1 to about 1:1.5.

5. The method according to claim 3, wherein the plant is from the Cannibas genus.

6. The method according to claim 3, wherein the saccharide comprises a disaccharide.

7. The method according to claim 6, wherein the disaccharide comprises sucrose.

8. The method according to claim 7, wherein the micropropagation media comprises about 0.1 to about 2.5 weight percent of sucrose, based on the total weight of the micropropagation media.

9. The method according to claim 3, wherein the micropropagation media further comprises at least one activated carbon.

10. The method according to claim 3, wherein the micropropagation media further comprises at least one cytokinin.

11. The method according to claim 3, wherein the sample is a meristem tissue.

12. A micropropagation media for facilitating plant cultivation, the micropropagation media comprising:
(a) water;
(b) a tissue culture medium;
(c) at least one gelling agent;
(d) at least one saccharide;
(e) a first auxin; and
(f) a second auxin, wherein the first auxin and the second auxin are different auxins, wherein the first auxin and the second auxin comprise indole-3-acetic acid (“IAA”), indole-3-butyric acid (“IBA”), naphthalene acetic acid (“NAA”), or 2,4-dichlorophenoxyacetic acid (“2,4-D”), wherein a molar ratio of the first auxin to the second auxin is in the range of about 3:1 to about 1:3.

13. The micropropagation media according to claim 12, wherein the first auxin comprises indole-3-butyric acid (“IBA”).

14. The micropropagation media according to claim 13, wherein the second auxin comprises naphthalene acetic acid (“NAA”).

15. The micropropagation media according to claim 14, wherein the molar ratio is in the range of about 1.5:1 to about 1:1.5.

16. The micropropagation media according to claim 15, wherein the saccharide comprises sucrose.

17. The micropropagation media according to claim 16, wherein the micropropagation media comprises about 0.1 to about 2.5 weight percent of sucrose, based on the total weight of the micropropagation media.

18. The micropropagation media according to claim 17, wherein the micropropagation media further comprises at least one activated carbon.

19. The micropropagation media according to claim 18, wherein the micropropagation media further comprises at least one cytokinin.

20. A method for facilitating Cannibas cultivation, the method comprising:
(a) providing a micropropagation media comprising—
(i) water,
(ii) a tissue culture medium,
(iii) at least one gelling agent,
(iv) 0.5 to 2.5 weight percent of sucrose based on the total weight of the micropropagation media,
(v) at least one activated carbon,
(vi) indole-3-butyric acid (“IBA”), and
(vii) naphthalene acetic acid (“NAA”), wherein a molar ratio of the IBA to the NAA is in the range of about 2:1 to about 1:2;
(b) providing a meristem sample from a Cannibas plant; and
(c) cultivating the meristem sample in at least a portion of the micropropagation media.
